(19) United States
(12) Patent Application Publication
(10) Pub. No.: US 2023/0197253 A1
(43) Pub. Date: Jun. 22, 2023

(54) MEDICAL IMAGE PROCESSING APPARATUS, METHOD, AND PROGRAM

(71) Applicant: FUJIFILM Corporation, Tokyo (JP)

(72) Inventors: Keigo NAKAMURA, Tokyo (JP); Akimichi ICHINOSE, Tokyo (JP)

(73) Assignee: FUJIFILM Corporation, Tokyo (JP)

(21) Appl. No.: 18/173,733

(22) Filed: Feb. 23, 2023

Related U.S. Application Data
(63) Continuation of application No. PCT/JP2021/023422, filed on Jun. 21, 2021.

(30) Foreign Application Priority Data
Sep. 29, 2020 (JP) 2020-163977

(51) Int. Cl. G16H 30/40; G06T 7/00; G06V 10/25

(52) U.S. Cl. CPC G16H 30/40; G06T 7/0012; G06V 10/25; G06T 2207/10072; G06T 2207/20104

(57) ABSTRACT

A medical image processing apparatus includes at least one processor, in which the processor is configured to: acquire a result of detecting at least one region of interest included in a medical image, which is detected by analyzing the medical image; specify a region of attention that a user has paid attention to in the medical image; specify a non-attention region of interest, which is a region of interest having a structure different from a structure related to the region of attention, among the regions of interest; and display a result of specifying the non-attention region of interest on a display.

### CROSS-REFERENCE TO RELATED APPLICATIONS

The present application is a Continuation of PCT International Application No. PCT/JP2021/023422, filed on Jun. 21, 2021, which claims priority to Japanese Patent Application No. 2020-163977, filed on Sep. 29, 2020. Each application above is hereby expressly incorporated by reference, in its entirety, into the present application.

### BACKGROUND

### Technical Filed

The present disclosure relates to a medical image processing apparatus, method, and program.

### Related Art

In recent years, advances in medical devices, such as computed tomography (CT) apparatuses and magnetic resonance imaging (MRI) apparatuses, have enabled image diagnosis using high-resolution medical images with higher quality. In particular, since a region of a lesion can be accurately specified by image diagnosis using CT images, MRI images, and the like, appropriate treatment is being performed based on the specified result.

In addition, image diagnosis is made by analyzing a medical image via computer-aided diagnosis (CAD) using a learning model in which machine learning is performed by deep learning or the like, and detecting diseased regions such as a lesion included in the medical image as regions of interest from the medical image. Here, a plurality of CAD learning models are prepared for each organ or each disease. Therefore, the CAD is configured to perform an analysis process that can detect all various diseases of various organs. In this way, the analysis result generated by the analysis process via CAD is saved in a database in association with examination information, such as a patient name, gender, age, and a modality which has acquired a medical image, and provided for diagnosis. A doctor interprets a medical image by referring to a distributed medical image and analysis result in his or her own interpretation terminal. At this time, in the interpretation terminal, an annotation is added to the region of interest including the disease included in the medical image based on the analysis result. For example, a region surrounding the region of interest, an arrow indicating the region of interest, a type and size of the disease, and the like are added as annotations. A radiologist refers to the annotations added to the region of interest to create an interpretation report.

On the other hand, the analysis result of the medical image via the above-described CAD is often used as a secondary interpretation (second reading) in the clinical field. For example, in interpreting a medical image, a doctor first interprets a medical image without referring to an analysis result via CAD. After that, the medical image to which the annotation is added based on the analysis result via CAD is displayed, and the doctor performs the secondary interpretation of the medical image while referring to the annotation. By performing such primary and secondary interpretations, it is possible to prevent the diseased region from being overlooked.

In addition, a method for efficiently performing primary interpretation and secondary interpretation has been proposed. For example, JP1992-333972A (JP-H04-333972A) and JP1994-259486A (JP-H06-259486A) propose a method in which an analysis result via CAD and an interpretation result via a doctor are compared and the interpretation result that the doctor has overlooked or read too much is presented to the doctor.

However, since the CAD is configured to perform an analysis process capable of detecting all the various diseases of the various organs, the analysis result via the CAD may include detection results of a large number of diseases as regions of interest. Here, in a case where an analysis result via CAD is displayed by using the methods described in JP1992-333972A (JP-H04-333972A) and JP1994-259486A (JP-H06-259486A), a region of interest interpreted by a doctor is displayed after being excluded from the analysis result. That is, the annotation is deleted and displayed for the region of interest that has been interpreted. However, in the methods described in JP1992-333972A (JP-H04-333972A) and JP1994-259486A (JP-H06-259486A), only the region of interest interpreted by the doctor is excluded from the analysis result via the CAD. Therefore, in the displayed medical image, since there are still many regions of interest to which the annotation is added, it is difficult to interpret the image with reference to the analysis result.

### SUMMARY OF THE INVENTION

The present disclosure has been made in view of the above circumstances, and an object of the present disclosure is to display analysis results for medical images in an easy-to-interpret manner.

According to an aspect of the present disclosure, there is provided a medical image processing apparatus comprising at least one processor, in which the processor is configured to: acquire a result of detecting at least one region of interest included in a medical image, which is detected by analyzing the medical image; specify a region of attention that a user has paid attention to in the medical image; specify a non-attention region of interest, which is a region of interest having a structure different from a structure related to the region of attention, among the regions of interest; and display a result of specifying the non-attention region of interest on a display.

Here, the “structure related to the region of attention” means a specific structure included in the medical image, and specifically, at least one of the disease or the organ can be a structure related to the region of attention.

In the medical image processing apparatus according to the aspect of the present disclosure, the processor may be configured to display the result of specifying the non-attention region of interest by erasing a result of detecting a region of interest having the structure related to the region of attention among the regions of interest.

In addition, in the medical image processing apparatus according to the aspect of the present disclosure, the processor may be configured to specify the region of attention based on an operation of the user during interpretation of the medical image.

In addition, in the medical image processing apparatus according to the aspect of the present disclosure, the processor may be configured to specify the region of attention based on a document regarding the medical image.

In addition, in the medical image processing apparatus according to the aspect of the present disclosure, the processor may be configured to specify the region of attention based on a method of displaying the medical image during interpretation of the medical image.

In addition, in the medical image processing apparatus according to the aspect of the present disclosure, the processor may be configured to display a result of detecting a region of interest having the structure related to the region of attention, the region of interest of which a feature amount derived at a time of detection is equal to or greater than a predetermined threshold value.

In addition, in the medical image processing apparatus according to the aspect of the present disclosure, the region of interest may be a region of interest for a plurality of types of diseases.

In addition, in the medical image processing apparatus according to the aspect of the present disclosure, the region of interest may be a region of interest for a plurality of types of organs.

According to another aspect of the present disclosure, there is provided a medical image processing method comprising: acquiring a result of detecting at least one region of interest included in a medical image, which is detected by analyzing the medical image; specifying a region of attention that a user has paid attention to in the medical image; specifying a non-attention region of interest, which is a region of interest having a structure different from a structure related to the region of attention, among the regions of interest; and displaying a result of specifying the non-attention region of interest on a display.

In addition, a program for causing a computer to execute the medical image processing method according to the aspect of the present disclosure may be provided.

According to the aspects of the present disclosure, it is possible to display analysis results for medical images in an easy-to-interpret manner.

### BRIEF DESCRIPTION OF THE DRAWINGS

FIG. 1 is a diagram showing a schematic configuration of a medical information system to which a medical image processing apparatus according to a first embodiment of the present disclosure is applied.

FIG. 2 is a diagram showing a schematic configuration of the medical image processing apparatus according to the first embodiment.

FIG. 3 is a functional configuration diagram of the medical image processing apparatus according to the first embodiment.

FIG. 4 is a diagram showing a result of detecting regions of interest by an analysis unit.

FIG. 5 is a diagram showing a display screen of a target medical image.

FIG. 6 is a diagram showing a result of specifying regions of attention by a radiologist for tomographic images.

FIG. 7 is a diagram showing a result of specifying non-attention regions of interest for the tomographic images.

FIG. 8 is a diagram showing a display screen of a result of specifying a non-attention region of interest according to the first embodiment.

FIG. 9 is a flowchart showing a process performed during primary interpretation in the first embodiment.

FIG. 10 is a flowchart showing a process performed during secondary interpretation in the first embodiment.

FIG. 11 is a diagram showing a display screen of a result of specifying a non-attention region of interest according to a second embodiment.

FIG. 12 is a diagram showing a display screen of a result of specifying a non-attention region of interest according to another embodiment.

FIG. 13 is a diagram showing a display screen of a result of specifying a non-attention region of interest according to another embodiment.

FIG. 14 is a diagram showing a display screen of a result of specifying a non-attention region of interest according to another embodiment.

FIG. 15 is a diagram showing a display screen of a result of specifying a non-attention region of interest according to another embodiment.

### DETAILED DESCRIPTION

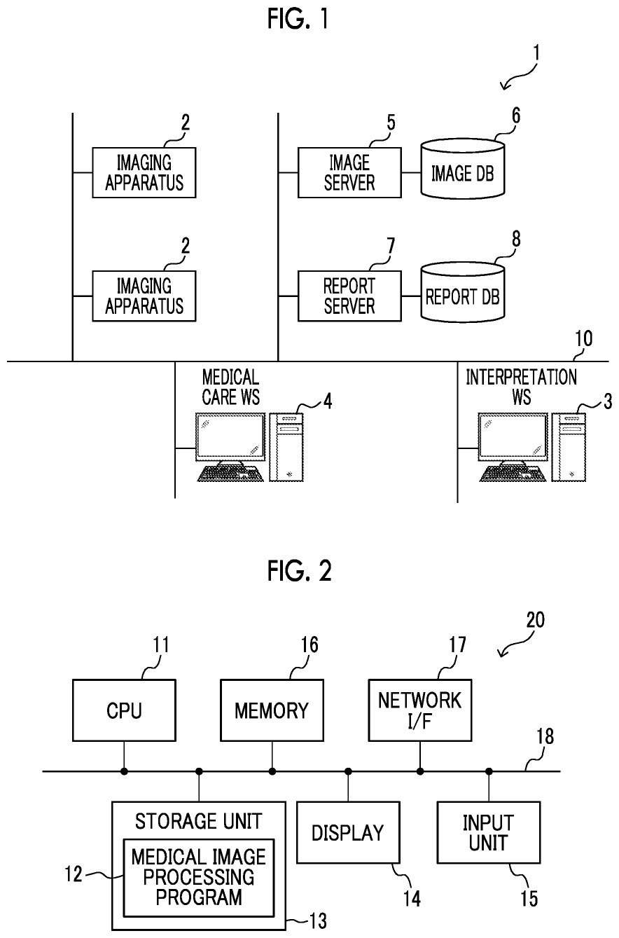

Hereinafter, embodiments of the present disclosure will be described with reference to the drawings. First, a configuration of a medical information system 1 to which a medical image processing apparatus according to the present embodiment is applied will be described. FIG. 1 is a diagram showing a schematic configuration of the medical information system 1. The medical information system 1 shown in FIG. 1 is, based on an examination order from a doctor in a medical department using a known ordering system, a system for imaging an examination target part of a subject, storing a medical image acquired by the imaging, interpreting the medical image by a radiologist and creating an interpretation report, and viewing the interpretation report and observing the medical image to be interpreted in detail by the doctor in the medical department that is a request source.

As shown in FIG. 1, in the medical information system 1, a plurality of imaging apparatuses 2, a plurality of interpretation workstations (WSs) 3 that are interpretation terminals, a medical care WS 4, an image server 5, an image database (hereinafter referred to as an image DB) 6, a report server 7, and a report database (hereinafter referred to as a report DB) 8 are communicably connected to each other through a wired or wireless network 10.

Each apparatus is a computer on which an application program for causing each apparatus to function as a component of the medical information system 1 is installed. The application program is stored in a storage apparatus of a server computer connected to the network 10 or in a network storage in a state in which it can be accessed from the outside, and is downloaded to and installed on the computer in response to a request. Alternatively, the application program is recorded on a recording medium, such as a digital versatile disc (DVD) and a compact disc read only memory (CD-ROM), and distributed, and is installed on the computer from the recording medium.

The imaging apparatus 2 is an apparatus (modality) that generates a medical image showing a diagnosis target part of the subject by imaging the diagnosis target part. Specifically, examples of the modality include a simple X-ray imaging apparatus, a CT apparatus, an MRI apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a radiologist of a radiology department to interpret a medical image and to create an interpretation report, and encompasses a medical image processing apparatus 20 according to a first embodiment. In the interpretation WS 3, a viewing request for a medical image to the image server 5, various image processing for the medical image received from the image server 5, display of the medical image, input reception of comments on findings regarding the medical image, and the like are performed. In the interpretation WS 3, creation of an interpretation report, a registration request and a viewing request for the interpretation report to the report server 7, display of the interpretation report received from the report server 7, and the like are performed. The above processes are performed by the interpretation WS 3 executing software programs for respective processes. The interpretation report is an example of a document regarding the medical image according to an aspect of the present disclosure.

The medical care WS 4 is a computer used by a doctor in a medical department to observe an image in detail, view an interpretation report, create an electronic medical record, and the like, and is configured to include a processing apparatus, a display apparatus such as a display, and an input apparatus such as a keyboard and a mouse. In the medical care WS 4, a viewing request for the image to the image server 5, display of the image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 comprises a storage in which the image DB 6 is configured. This storage may be a hard disk apparatus connected to the image server 5 by a data bus, or may be a disk apparatus connected to a storage area network (SAN) or a network attached storage (NAS) connected to the network 10. In a case where the image server 5 receives a request to register a medical image from the imaging apparatus 2, the image server 5 prepares the medical image in a format for a database and registers the medical image in the image DB 6.

Image data of the medical image acquired by the imaging apparatus 2 and accessory information are registered in the image DB 6. The accessory information includes, for example, an image identification (ID) for identifying each medical image, a patient ID for identifying a subject, an examination ID for identifying an examination, a unique ID (unique identification (UID)) allocated for each medical image, examination date and examination time at which a medical image is generated, the type of imaging apparatus used in an examination for acquiring a medical image, patient information such as the name, age, and gender of a patient, an examination part (an imaging part), imaging information (an imaging protocol, an imaging sequence, an imaging method, imaging conditions, the use of a contrast medium, and the like), and information such as a series number or a collection number in a case where a plurality of medical images are acquired in one examination.

In addition, in a case where the viewing request from the interpretation WS 3 and the medical care WS 4 is received through the network 10, the image server 5 searches for a medical image registered in the image DB 6 and transmits the searched for medical image to the interpretation WS 3 and to the medical care WS 4 that are request sources.

The report server 7 incorporates a software program for providing a function of a database management system to a general-purpose computer. In a case where the report server 7 receives a request to register the interpretation report from the interpretation WS 3, the report server 7 prepares the interpretation report in a format for a database and registers the interpretation report in the report DB 8.

In the report DB 8, an interpretation report created by the radiologist using the interpretation WS 3 is registered. The interpretation report may include information such as, for example, a medical image to be interpreted, an image ID for identifying the medical image, a radiologist ID for identifying the radiologist who performed the interpretation, a disease name, disease position information, and information for accessing a medical image.

Further, in a case where the report server 7 receives the viewing request for the interpretation report from the interpretation WS 3 and the medical care WS 4 through the network 10, the report server 7 searches for the interpretation report registered in the report DB 8, and transmits the searched for interpretation report to the interpretation WS 3 and to the medical care WS 4 that are request sources.

In the present embodiment, it is assumed that the diagnosis target is a thoracoabdominal region of a human body, the medical image is a three-dimensional CT image consisting of a plurality of tomographic images including the thoracoabdominal region, and an interpretation report including comments on findings for diseases such as lungs and livers included in the thoracoabdominal region is created by interpreting the CT image. The medical image is not limited to the CT image, and any medical image such as an MRI image and a simple two-dimensional image acquired by a simple X-ray imaging apparatus can be used.

In the present embodiment, in creating the interpretation report, the radiologist first displays a medical image on a display 14 and interprets the medical image with his/her own eyes. After that, a region of interest included in the medical image is detected by analyzing the medical image with the medical image processing apparatus according to the present embodiment, and a second interpretation is performed using the detection result. The first interpretation is referred to as a primary interpretation, and the second interpretation using the result of detecting the region of interest by the medical image processing apparatus according to the present embodiment is referred to as a secondary interpretation.

The network 10 is a wired or wireless local area network that connects various apparatuses in a hospital to each other. In a case where the interpretation WS 3 is installed in another hospital or clinic, the network 10 may be configured to connect local area networks of respective hospitals through the Internet or a dedicated line.

Next, the medical image processing apparatus according to the first embodiment will be described. FIG. 2 describes a hardware configuration of the medical image processing apparatus according to the first embodiment. As shown in FIG. 2, the medical image processing apparatus 20 includes a central processing unit (CPU) 11, a non-volatile storage 13, and a memory 16 as a temporary storage area. Further, the medical image processing apparatus 20 includes a display 14 such as a liquid crystal display, an input device 15 consisting of a pointing device such as a keyboard and a mouse, and a network interface (I/F) 17 connected to the network 10. The CPU 11, the storage 13, the display 14, the input device 15, the memory 16, and the network I/F 17 are connected to a bus 18. The CPU 11 is an example of a processor in the present disclosure.

The storage 13 is realized by a hard disk drive (HDD), a solid state drive (SSD), a flash memory, and the like. A medical image processing program 12 is stored in the storage 13 as the storage medium. The CPU 11 reads out the medical image processing program 12 from the storage 13, loads the read program into the memory 16, and executes the loaded medical image processing program 12.

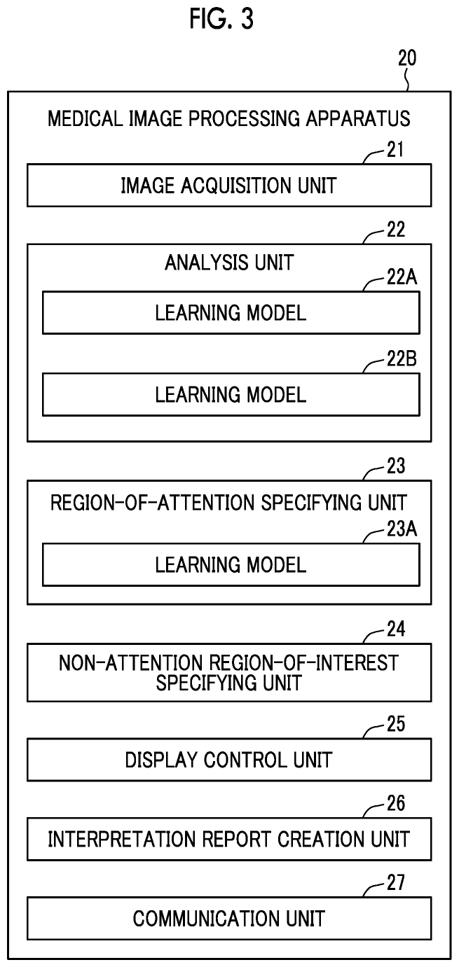

Next, a functional configuration of the medical image processing apparatus according to the first embodiment will be described. FIG. 3 is a diagram showing a functional configuration of the medical image processing apparatus according to the first embodiment. As shown in FIG. 3, the medical image processing apparatus 20 comprises an information acquisition unit 21, an analysis unit 22, a region-of-attention specifying unit 23, a non-attention region-of-interest specifying unit 24, a display control unit 25, an interpretation report creation unit 26, and a communication unit 27. Then, the CPU 11 executes the medical image processing program 12, so that the CPU 11 functions as the information acquisition unit 21, the analysis unit 22, the region-of-attention specifying unit 23, the non-attention region-of-interest specifying unit 24, the display control unit 25, the interpretation report creation unit 26, and the communication unit 27.

The information acquisition unit 21 acquires a target medical image GO to be processed for creating an interpretation report from the image server 5 according to an instruction from the input device 15 by the radiologist who is an operator. The target medical image GO is, for example, a three-dimensional CT image consisting of a plurality of tomographic images acquired by imaging a thoracoabdominal region of a human body. In addition, in a case where an interpretation report has already been created and registered in the report DB 8 for the target medical image GO, the information acquisition unit 21 acquires the interpretation report from the report server 7, as necessary.

The analysis unit 22 detects a region of the abnormal shadow included in the target medical image GO as a region of interest, and derives an annotation for the detected region of interest. The analysis unit 22 detects regions of shadows of a plurality of types of diseases as regions of interest from the target medical image GO using a known computer-aided diagnosis (that is, CAD) algorithm, derives properties of the detected regions of interest, and derives annotations based on the properties.

Examples of the type of the disease include a tumor, pleural effusion, nodule, calcification, fracture, and the like, depending on the part of the subject included in the target medical image GO. Note that the analysis unit 22 detects the region of the abnormal shadow included in the plurality of types of organs included in the target medical image GO as the region of interest. In the present embodiment, since the target medical image GO includes the thoracoabdominal region of the human body, examples of organs include various organs included in the thoracoabdominal region of the human body, such as lungs, heart, liver, stomach, small intestine, pancreas, spleen, and kidneys.

In order to detect the regions of interest and derive the annotations, the analysis unit 22 has a learning model 22A in which machine learning is performed to detect shadows of a plurality of types of diseases as the regions of interest from the target medical image GO, and derive properties. Further, the analysis unit 22 has a learning model 22B that derives an annotation by documenting the properties derived by the learning model 22A.

A plurality of learning models 22A are prepared according to the type of disease and the type of organ. The learning model 22A consists of a convolutional neural network (CNN) in which deep learning has been performed using supervised training data so as to discriminate whether or not each pixel (voxel) in the target medical image GO represents a shadow of various diseases or an abnormal shadow.

The learning model 22A is constructed by training CNN using, for example, a large amount of supervised training data consisting of supervised training images that include abnormal shadows, a region of the abnormal shadows in the supervised training image, and correct answer data representing the properties of the abnormal shadows, and a large amount of supervised training data consisting of supervised training images that do not include abnormal shadows. The learning model 22A derives a degree of certainty (likelihood) indicating that each pixel in the medical image is an abnormal shadow, and detects a region consisting of pixels whose degree of certainty is equal to or higher than a predetermined first threshold value as a region of interest. Here, the degree of certainty is a value of 0 or more and 1 or less. Further, the learning model 22A derives the properties of the detected region of interest. The properties include the position and the size of the abnormal shadow, the type of disease, and the like. The type of the disease includes a nodule, mesothelioma, a calcification, a pleural effusion, a tumor, a cyst, and the like.

The learning model 22A may detect an abnormal shadow from a three-dimensional medical image, or may detect an abnormal shadow from each of a plurality of tomographic images constituting the target medical image GO.

Further, as the learning model 22A, any learning model such as, for example, a support vector machine (SVM) can be used in addition to the convolutional neural network.

The learning model 22B derives an annotation based on the properties derived by the learning model 22A. The learning model 22B consists of, for example, a recurrent neural network in which machine learning is performed to document the input properties. Assuming that the properties derived by the learning model 22A are “upper lobe of the left lung”, “nodule”, and “1 cm”, the learning model 22B derives the sentence “A nodule of 1 cm in size is seen in the upper lobe of the left lung” as an annotation.

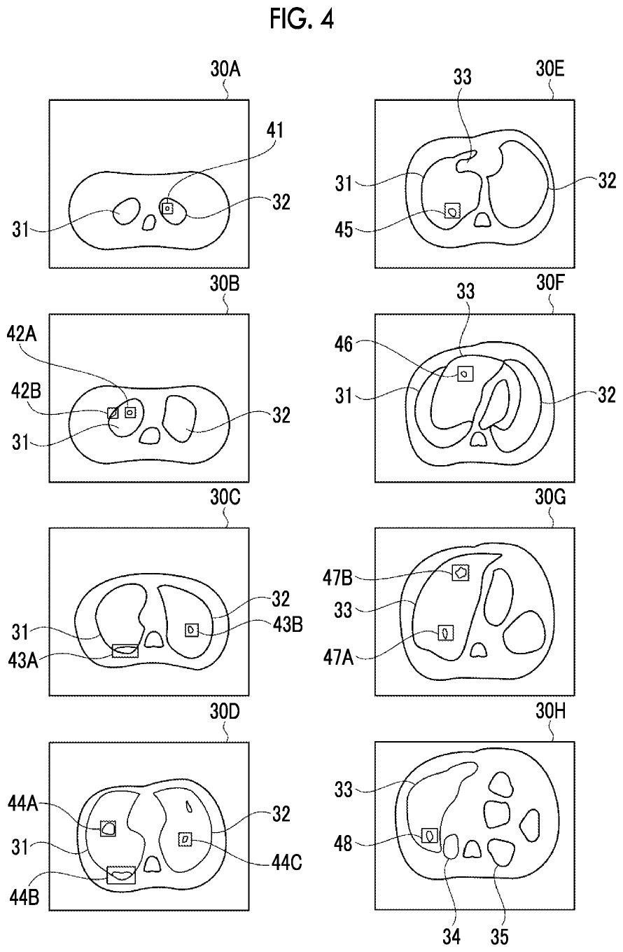

FIG. 4 is a diagram showing result of detecting regions of interest by the analysis unit 22. In the present embodiment, the target medical image GO is a CT image of the thoracoabdominal region of a human body and consists of tomographic images of a plurality of axial cross sections. In FIG. 4, eight tomographic images 30A to 30H are shown in order from the head side of the human body. The tomographic images 30A to 30F include a left lung 31 and a right lung 32. The tomographic images 30E to 30H include a liver 33. The tomographic image 30H includes a left kidney 34 and a right kidney 35.

In FIG. 4, the abnormal shadow detected in each tomographic image is surrounded by a rectangular mark. That is, as shown in FIG. 4, in the tomographic image 30A, a nodule region of the right lung 32 surrounded by a mark 41 is detected as a region of interest. In the tomographic image 30B, a nodule region of the left lung 31 surrounded by a mark 42A and a mesothelioma region of the left lung 31 surrounded by a mark 42B are detected as regions of interest. In the tomographic image 30C, a pleural effusion region of the left lung 31 surrounded by a mark 43A is detected as a region of interest, and a nodule region of the right lung 32 surrounded by a mark 43B is detected as a region of interest. In the tomographic image 30D, a nodule region of the left lung 31 surrounded by a mark 44A and a pleural effusion region of the left lung 31 surrounded by a mark 44B are detected as regions of interest, and a calcification region of the right lung 32 surrounded by a mark 44C is detected as a region of interest. Note that the nodule region of the right lung 32 is omitted from detection. In the tomographic image 30E, a nodule region of the left lung 31 surrounded by a mark 45 is detected as a region of interest. In the tomographic image 30F, a tumor region of the liver 33 surrounded by a mark 46 is detected as a region of interest. In the tomographic image 30G, two tumor regions of the liver 33 surrounded by marks 47A and 47B are detected as regions of interest. In the tomographic image 30H, a cyst region of the liver 33 surrounded by a mark 48 is detected as a region of interest. Note that the tomographic images 30A to 30H shown in FIG. 4 show a state in which many regions of interest are detected for the sake of description, and are different from the actual appearance of the regions of interest in the human body.

The analysis unit 22 also derives annotations for the detected regions of interest. For example, with respect to the tomographic image 30C, the analysis unit 22 derives the annotations “pleural effusion in the posterior part of the middle lobe of the left lung” and “nodule of 1 cm in size in the middle lobe of the right lung”. In addition, the analysis unit 22 derives the annotation of “tumor of 1 cm in size in the liver” with respect to the tomographic image 30F.

In a case where the display control unit 25 displays the result of detecting the region of interest detected by the analysis unit 22 in this way and the derived annotation (hereinafter simply referred to as the analysis result) on the display 14 as described later, a mark is added to the region of interest detected by the analysis unit 22 and an annotation is displayed.

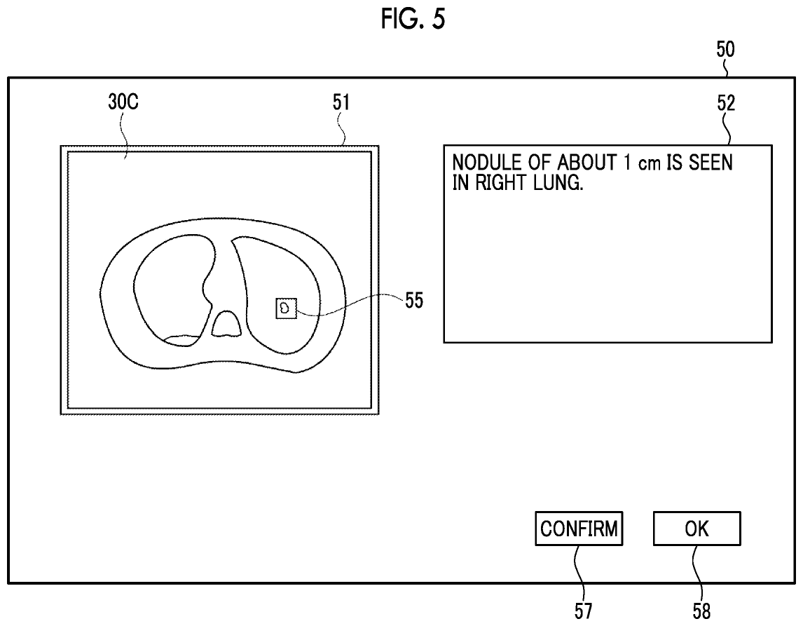

The region-of-attention specifying unit 23 specifies a region of attention that the radiologist has paid attention to in the target medical image GO. Specifically, the radiologist displays the target medical image GO on the display 14 as the primary interpretation, interprets the target medical image GO with his/her own eyes, and specifies a region of the found abnormal shadow as a region of attention. FIG. 5 is a diagram showing a display screen of a target medical image. As shown in FIG. 5, a display screen 50 includes an image display region 51 and a sentence display region 52. In the image display region 51, tomographic images representing tomographic planes of the target medical image GO are displayed in a switchable manner. In FIG. 5, the tomographic image 30C shown in FIG. 4 is displayed in the image display region 51. In addition, in the sentence display region 52, a comment on findings by the radiologist who interpreted the displayed tomographic image is described. The comment on findings is also an example of a document regarding the medical image.

The radiologist can switch the tomographic image displayed in the image display region 51 by using the input device 15. Also, the input device 15 can add a mark to the abnormal shadow included in the tomographic image and measure the size of the abnormal shadow. The region-of-attention specifying unit 23 specifies the region of the abnormal shadow to which the mark is added as the region of attention. As the mark, a rectangle surrounding the abnormal shadow, an arrow indicating the abnormal shadow, or the like can be used. In FIG. 5, a rectangular mark 55 is added to the nodule included in the right lung of the tomographic image 30C displayed in the image display region 51.

Note that even though the radiologist does not add the mark, it can be considered that the interpretation of the abnormal shadow whose size has been measured has been completed. Therefore, the region-of-attention specifying unit 23 also specifies the region of the abnormal shadow whose size has been measured as the region of attention.

In addition, the radiologist can input the comment on findings for the target medical image GO to the sentence display region 52 by using the input device 15. In FIG. 5, the comment on findings “A nodule of about 1 cm is seen in the right lung” is described in the sentence display region 52.

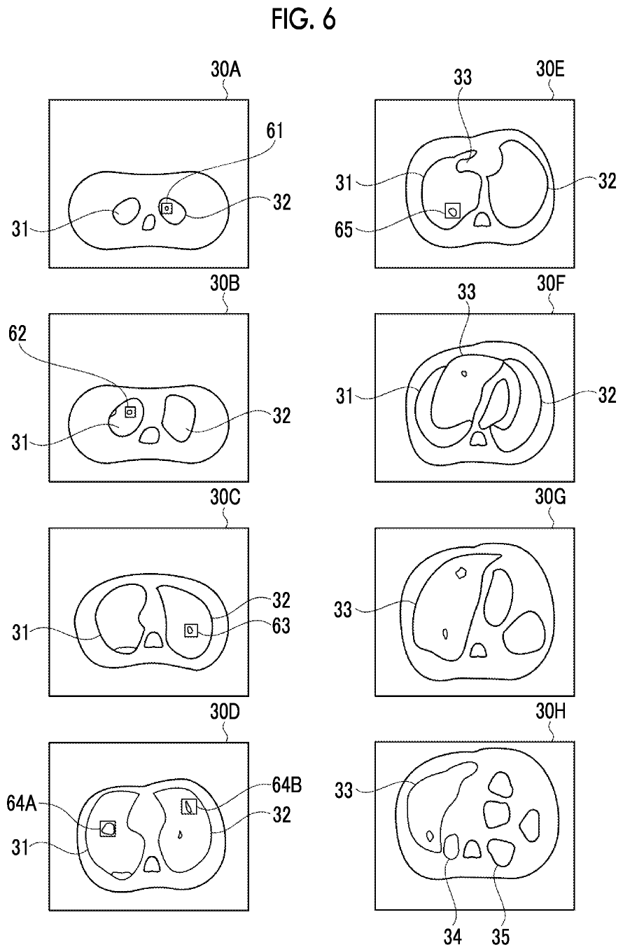

FIG. 6 is a diagram showing a result of specifying regions of attention by a radiologist for tomographic images. As shown in FIG. 6, in the tomographic image 30A, a nodule region of the right lung 32 surrounded by a mark 61 is specified as a region of attention. In the tomographic image 30B, a nodule region of the left lung 31 surrounded by a mark 62 is specified as a region of attention. In the tomographic image 30C, a nodule region of the right lung 32 surrounded by a mark 63 is specified as a region of attention. In the tomographic image 30D, a nodule region of the left lung 31 surrounded by a mark 64A and a nodule region of the right lung 32 surrounded by a mark 64B are specified as regions of attention. Note that the nodule region of the right lung 32 is a region that has been omitted from the result of detecting the region of interest by the analysis unit 22. In the tomographic image 30E, a nodule region of the left lung 31 surrounded by a mark 65 is specified as a region of attention. Note that no region of attention is specified in the tomographic images 30F to 30H.

After completion of the primary interpretation, the radiologist selects a confirmation button 57 on the display screen 50. Accordingly, the secondary interpretation is started. In the secondary interpretation, the non-attention region-of-interest specifying unit 24 specifies the non-attention region of interest among the regions of interest detected by the analysis unit 22. The non-attention region of interest is a region of interest having a structure different from the structure related to the above-described region of attention. The structure can be at least one of a disease that is the region of attention or an organ that includes the region of attention. In the first embodiment, it is assumed that the non-attention region of interest is a region of interest for an organ different from the organ related to the region of attention. In addition, in the present embodiment, the non-attention region-of-interest specifying unit 24 specifies, as a non-attention region of interest, a region of interest detected in an organ for which the radiologist did not specify the region of attention during primary interpretation.

Here, comparing FIG. 4 showing the analysis result by the analysis unit 22 with FIG. 6 which is the interpretation result by the radiologist, a region of attention is not specified in the liver in the interpretation result by the radiologist. Therefore, the non-attention region-of-interest specifying unit 24 specifies the region of interest specified by the analysis unit 22 in the liver as the non-attention region of interest. That is, as shown in FIG. 4, the non-attention region-of-interest specifying unit 24 specifies, as non-attention regions of interest, the tumor region of the liver 33 surrounded by the mark 46 in the tomographic image 30F, the tumor regions of the liver 33 surrounded by the marks 47A and 47B in the tomographic image 30G, and the cyst region of the liver 33 surrounded by the mark 48 in the tomographic image 30H.

In addition, in the present embodiment, as shown in FIG. 6, for the tomographic image 30B, the region-of-attention specifying unit 23 does not specify the mesothelioma included in the left lung 31 (the mesothelioma surrounded by the mark 42B in the tomographic image 30B shown in FIG. 4) as the region of attention. Also, for the tomographic image 30C, the region-of-attention specifying unit 23 does not specify the pleural effusion included in the left lung 31 (the pleural effusion surrounded by the mark 43A in the tomographic image 30C shown in FIG. 4) as the region of attention. In addition, for the tomographic image 30D, the region-of-attention specifying unit 23 does not specify the pleural effusion included in the left lung 31 (the pleural effusion surrounded by the mark 44B in the tomographic image 30D shown in FIG. 4) and the calcification included in the right lung 32 (the calcification surrounded by the mark 44C in the tomographic image 30D shown in FIG. 4) as the regions of attention. However, diseases not specified as regions of attention in the lung will be described later.

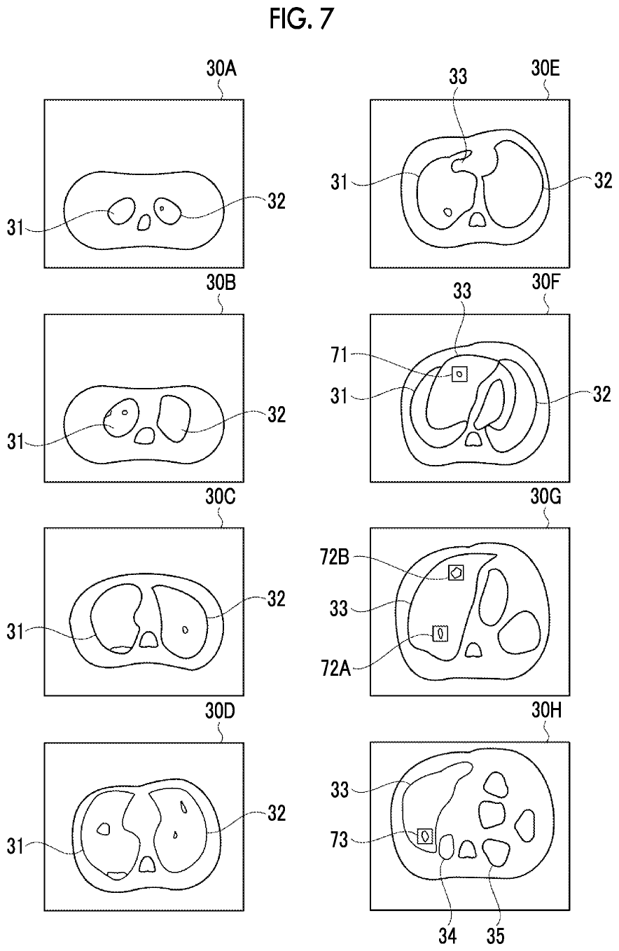

FIG. 7 is a diagram showing a result of specifying non-attention regions of interest for the tomographic images. Note that FIG. 7 shows the tomographic images displayed on the display 14. Therefore, in FIG. 7, among the regions of interest detected by the analysis unit 22, the marks added to the regions of attention as shown in FIG. 4 are erased and the marks are added to only the non-attention regions of interest. As shown in FIG. 7, in the tomographic images 30A to 30E, the non-attention region of interest is not specified. In the tomographic image 30F, a tumor region of the liver 33 surrounded by a rectangular mark 71 is specified as a non-attention region of interest. In the tomographic image 30G, tumor regions of the liver 33 surrounded by rectangular marks 72A and 72B are specified as non-attention regions of interest. In the tomographic image 30H, a cyst region of the liver 33 surrounded by a rectangular mark 73 is specified as a non-attention region of interest.

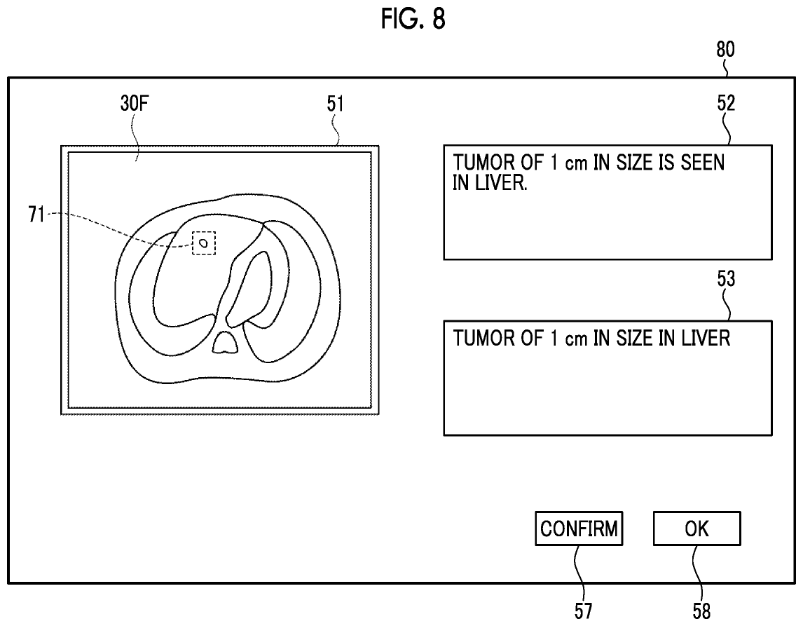

The display control unit 25 displays the result of specifying the non-attention regions of interest on the display 14. FIG. 8 is a diagram showing a display screen of a result of specifying a non-attention region of interest. The result of specifying the non-attention region of interest is a mark added to the non-attention region of interest and an annotation derived for the non-attention region of interest. In FIG. 8, the same reference numerals are assigned to the same configurations as those in FIG. 5, and detailed description thereof will be omitted here.

As shown in FIG. 8, the tomographic image 30F is displayed in the image display region 51 of a display screen 80 of the result of specifying the non-attention region of interest. In the tomographic image 30F, the rectangular mark 71 is added to the tumor of the liver which is a non-attention region of interest.

In addition, an annotation display region 53 that displays annotations for the non-attention region of interest is displayed on the display screen 80. As shown in FIG. 8, in the annotation display region 53, an annotation of “tumor of 1 cm in size in the liver” derived by the analysis unit 22 for the tomographic image 30F is displayed.

In a case where the tomographic images 30A to 30E in which all the regions of interest detected by the analysis unit 22 are specified as regions of attention are displayed in the image display region 51, no mark is added to the abnormal shadow and no annotation is displayed. On the other hand, the radiologist does not specify an abnormal shadow included in the liver as a region of attention. Therefore, in a case where the tomographic images 30F to 30H in which the region of interest is detected in the liver are displayed in the image display region 51, a mark is added to the abnormal shadow included in the liver and an annotation is displayed.

The radiologist can confirm the presence or absence of the abnormal shadow that may have been overlooked during the primary interpretation through the mark added to the non-attention region of interest and the displayed annotation. For example, as shown in FIG. 8, the mark 71 is added to the tumor included in the liver included in the tomographic image 30F, and the annotation is displayed. Accordingly, the radiologist can easily confirm the presence or absence of the tumor included in the liver that was overlooked during the primary interpretation, and can describe a comment on findings in the sentence display region 52 for the confirmed tumor. For example, in FIG. 8, it is possible to describe the comment on findings “A tumor of about 1 cm is seen in the liver”.

The interpretation report creation unit 26 creates an interpretation report including the comment on findings input to the sentence display region 52. Then, in a case where an OK button 58 is selected on the display screen 80, the interpretation report creation unit 26 saves the created interpretation report together with the target medical image GO and the detection result in the storage 13.

The communication unit 27 transfers the created interpretation report together with the target medical image GO and the detection result to the report server 7. In the report server 7, the transferred interpretation report is saved together with the target medical image GO and the detection result.

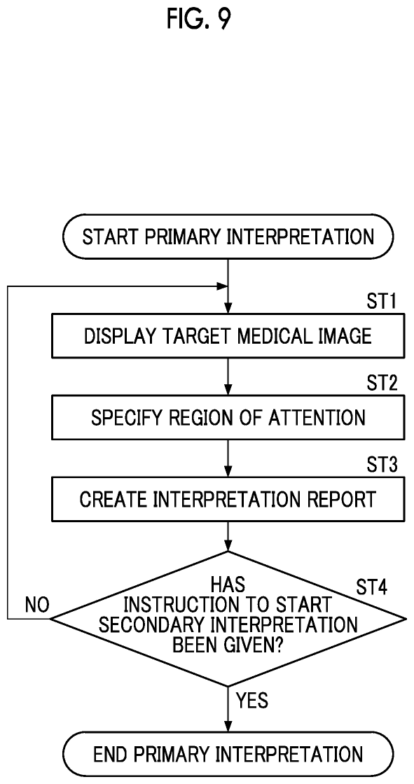

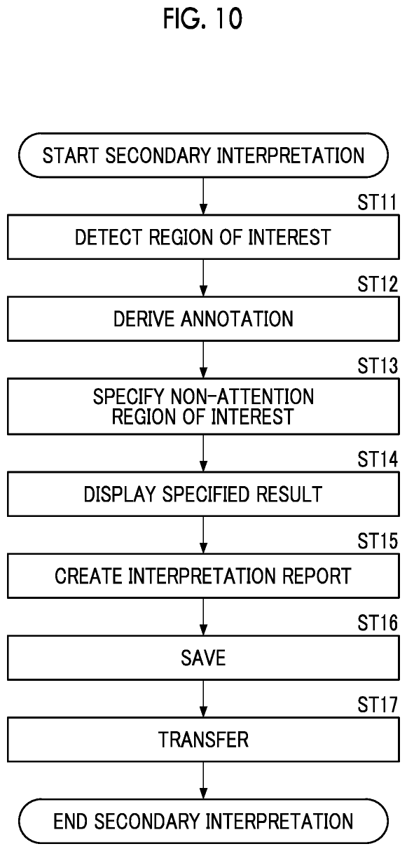

Next, a process performed in the first embodiment will be described. FIG. 9 is a flowchart showing a process performed during the primary interpretation in the first embodiment, and FIG. 10 is a flowchart showing a process performed during the secondary interpretation in the first embodiment. It is assumed that the target medical image GO to be interpreted is acquired from the image server 5 by the information acquisition unit 21 and is saved in the storage 13. The process is started in a case where the radiologist issues an instruction to create an interpretation report, and the display control unit 25 displays the target medical image GO on the display 14 (Step ST1). Next, the region-of-attention specifying unit 23 specifies a region of attention that the radiologist has paid attention to in the target medical image GO based on an instruction from the radiologist using the input device 15 (Step ST2). The radiologist inputs comments on findings regarding the region of attention into the sentence display region 52.

Subsequently, the interpretation report creation unit 26 creates an interpretation report based on the primary interpretation using the comments on findings input by the radiologist into the sentence display region 52 (Step ST3). Next, by selecting the confirmation button 57, it is determined whether or not the instruction to start the secondary interpretation has been given (Step ST4), and in a case where determination in Step ST4 is negative, the process returns to Step ST1. In a case where determination in Step ST4 is affirmative, the primary interpretation is terminated and the secondary interpretation is started.

During the secondary interpretation, first, the analysis unit 22 analyzes the target medical image GO to detect at least one region of interest included in the target medical image GO (Step ST11). Also, an annotation for the region of interest is derived (Step ST12). The analysis of the target medical image GO may be performed immediately after the target medical image GO is acquired from the image server 5 via the information acquisition unit 21.

Next, the non-attention region-of-interest specifying unit 24 specifies a non-attention region of interest that is a region of interest for an organ different from the organ related to the region of attention among the regions of interest detected by the analysis unit 22 (Step ST13). Then, the display control unit 25 displays the result of specifying the non-attention region of interest on the display 14 (Step ST14). The radiologist inputs the comment on findings into the sentence display region 52, as necessary, while observing the result of specifying the non-attention region of interest.

Next, the interpretation report creation unit 26 creates an interpretation report using the comments on findings input by the radiologist (Step ST15). Then, the interpretation report creation unit 26 saves the created interpretation report together with the target medical image GO and the detection result in the storage 13 (Step ST16). Further, the communication unit 27 transfers the created interpretation report together with the target medical image GO and the detection result to the report server 7 (Step ST17), and ends the process of the secondary interpretation.

In this way, in the first embodiment, a region of attention that the radiologist who is the user has paid attention to in the target medical image GO is specified, a non-attention region of interest that is a region of interest for an organ different from the organ related to the region of attention among the regions of interest detected by the analysis unit 22 from the target medical image GO is specified, and the result of specifying the non-attention region of interest is displayed on the display 14. Accordingly, instead of the extraction results of all the regions of interest detected by the analysis unit 22, only the regions of interest detected in the organ for which the radiologist has not specified the regions of attention are displayed on the display 14 as the non-attention regions of interest. Therefore, the analysis results for the target medical image GO can be reduced, and thus the analysis results for the target medical image GO can be displayed in an easy-to-interpret manner.

Next, a second embodiment of the present disclosure will be described. The configuration of a medical image processing apparatus according to the second embodiment is the same as the configuration of the medical image processing apparatus according to the first embodiment shown in FIG. 3, and only the processing to be performed is different. Therefore, detailed description of the apparatus will be omitted here.

In the first embodiment, the non-attention region-of-interest specifying unit 24 specifies a non-attention region of interest that is a region of interest for an organ different from the organ related to the region of attention among the regions of interest detected by the analysis unit 22 from the target medical image GO. The second embodiment differs from the first embodiment in that the non-attention region-of-interest specifying unit 24 specifies, as a non-attention region of interest, a region of interest for a disease different from the disease related to the region of attention among the regions of interest detected by the analysis unit 22 from the target medical image GO.

For example, in a case where the tomographic image 30B is interpreted, as shown in FIG. 6, the radiologist does not specify the mesothelioma included in the left lung 31 (the mesothelioma surrounded by the mark 42B in the tomographic image 30B shown in FIG. 4) as the region of attention. Also, for the tomographic image 30C, the radiologist does not specify the pleural effusion included in the left lung 31 (the pleural effusion surrounded by the mark 43A in the tomographic image 30C shown in FIG. 4) as the region of attention. In addition, for the tomographic image 30D, the radiologist does not specify the pleural effusion included in the left lung 31 (the pleural effusion surrounded by the mark 44B in the tomographic image 30D shown in FIG. 4) and the calcification included in the right lung 32 (the calcification surrounded by the mark 44C in the tomographic image 30D shown in FIG. 4) as the regions of attention. In such a case, the radiologist may have overlooked the mesothelioma and pleural effusion included in the left lung 31, and the calcification included in the right lung 32.

Therefore, in the second embodiment, the non-attention region-of-interest specifying unit 24 specifies the region of interest for a disease different from the disease related to the region of attention as the non-attention region of interest. Here, the disease related to the region of attention is the nodule, and the different diseases are mesothelioma, pleural effusion, and calcification. The non-attention region-of-interest specifying unit 24 specifies, for the tomographic image 30B, the region of interest of the mesothelioma included in the left lung 31 as the non-attention region of interest. In addition, the non-attention region-of-interest specifying unit 24 specifies, for the tomographic image 30C, the region of interest of the pleural effusion included in the left lung 31 as the non-attention region of interest. In addition, the non-attention region-of-interest specifying unit 24 specifies, for the tomographic image 30D, the region of interest of the pleural effusion included in the left lung 31 and the region of interest of the calcification included in the right lung 32 as the non-attention regions of interest.

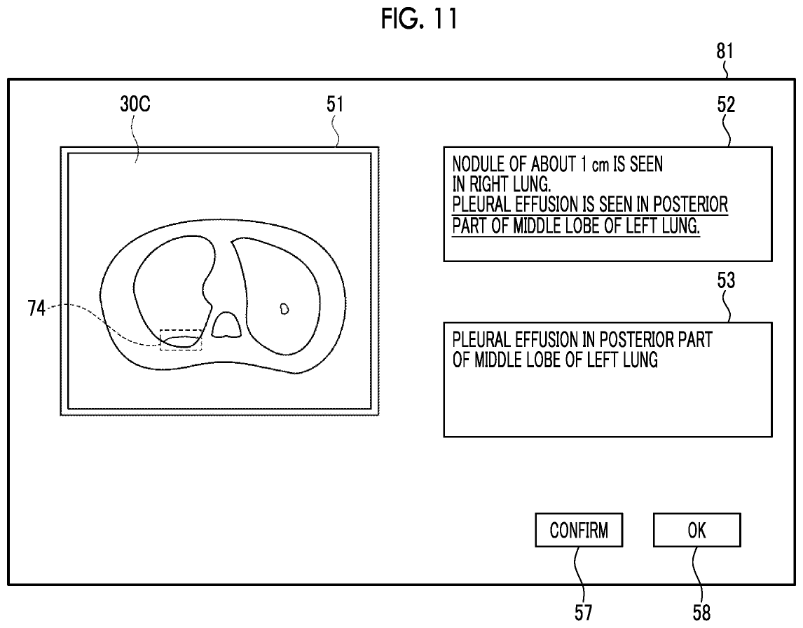

Accordingly, as shown in FIG. 11, in a case where the tomographic image 30C is displayed in the image display region 51 on the display screen 81 of the result of specifying the non-attention region of interest, the display control unit 25 adds a rectangular mark 74 to the pleural effusion included in the left lung 31, and displays the annotation “pleural effusion in the posterior part of the middle lobe of the left lung” derived for the pleural effusion in the annotation display region 53. In the tomographic image 30C displayed in the image display region 51, the mark 43B added as shown in FIG. 4 is erased. On the other hand, in a case where the tomographic image 30B is displayed on the display screen 81 of the result of specifying the non-attention region of interest, the display control unit 25 adds a mark to the mesothelioma included in the left lung 31, and displays, in the annotation display region 53, the annotation for the mesothelioma in the left lung derived by the analysis unit 22 for the tomographic image 30B. In the tomographic image 30B displayed in the image display region 51, the mark 42A added as shown in FIG. 4 is erased. In addition, in a case where the tomographic image 30D is displayed on the display screen 81 of the result of specifying the non-attention region of interest, the display control unit 25 adds marks to the pleural effusion included in the left lung 31 and the calcification included in the right lung 32, and displays, in the annotation display region 53, the annotations for the pleural effusion included in the left lung and the calcification included in the right lung derived by the analysis unit 22 for the tomographic image 30D. In the tomographic image 30D displayed in the image display region 51, the marks 44A and 44C added as shown in FIG. 4 are erased.

The radiologist can confirm that a pleural effusion is present in the left lung by confirming the annotation displayed in the mark 74 and the annotation display region 53 in the tomographic image 30C displayed on the display screen 81 shown in FIG. 11. Therefore, the radiologist can additionally write the comment on findings “A pleural effusion is seen in the posterior part of the middle lobe of the left lung” to the comment on findings “A nodule of about 1 cm is seen in the right lung” described in the sentence display region 52.

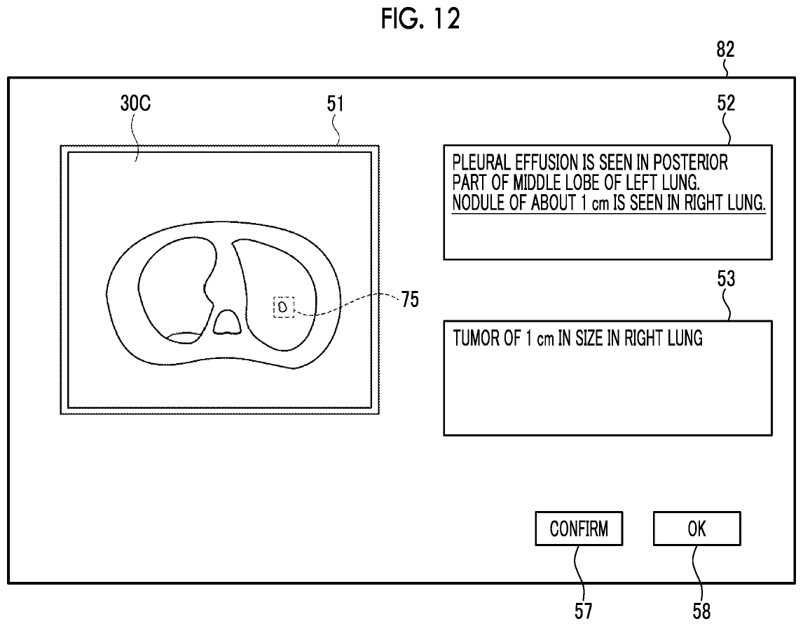

Contrary to the above, in a case where the region of interest of the nodule in the right lung detected by the analysis unit 22 is not specified as a region of attention in the tomographic image 30C, in the second embodiment, the non-attention region-of-interest specifying unit 24 specifies, as a non-attention region of interest, the region of interest of the nodule in the right lung among the regions of interest detected by the analysis unit 22. In this case, as shown in FIG. 12, in a case where the tomographic image 30C is displayed in the image display region 51 on the display screen 82 of the result of specifying the non-attention region of interest, a rectangular mark 75 is added to the abnormal shadow of the nodule in the right lung. In addition, in the annotation display region 53, “nodule of 1 cm in size in the right lung”, which is an annotation for the nodule in the right lung derived by the analysis unit 22 for the tomographic image 30C, is displayed. Therefore, the radiologist can additionally write the comment on findings “A nodule of about 1 cm is seen in the right lung” to the comment on findings “A pleural effusion is seen in the posterior part of the middle lobe of the left lung” described in the sentence display region 52.

In this way, in the second embodiment, a region of attention that the radiologist who is the user has paid attention to in the target medical image GO is specified, a non-attention region of interest that is a region of interest for a disease different from the disease related to the region of attention among the regions of interest detected by the analysis unit 22 from the target medical image GO is specified, and the result of specifying the non-attention region of interest is displayed on the display 14. Accordingly, instead of the extraction results of all the regions of interest detected by the analysis unit 22, only the regions of interest related to the disease for which the radiologist has not specified the regions of attention are displayed on the display 14 as the non-attention regions of interest. Therefore, the analysis results for the target medical image GO can be reduced, and the analysis results for the target medical image GO can be displayed in an easy-to-interpret manner.

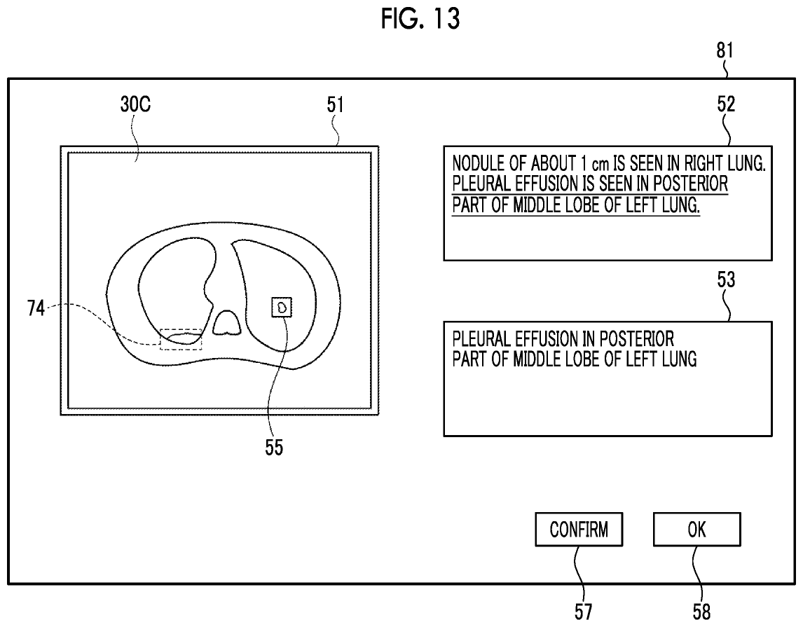

In the first and second embodiments, the mark is added only to the non-attention region of interest on the display screen 81 of the result of specifying the non-attention region of interest, but the present disclosure is not limited thereto. Different marks may be added to each of the region of attention and the non-attention region of interest. For example, as shown in FIG. 6, in the tomographic image 30C, in a case where the nodule included in the right lung 32 is specified as a region of attention, the pleural effusion included in the left lung 31 is specified as a non-attention region of interest. In this case, as shown in FIG. 13, in a case where the tomographic image 30C is displayed on the display screen 81 of the result of specifying the non-attention region of interest, a solid rectangular mark 55 may be added to the nodule included in the right lung 32 and a dashed rectangular mark 74 may be added to the pleural effusion included in the left lung 31.

In addition, in the first and second embodiments, the region-of-attention specifying unit 23 specifies the region of attention based on the fact that the radiologist specifies the abnormal shadow included in the target medical image GO, but the present disclosure is not limited thereto. The radiologist may specify the region of attention included in the target medical image GO based on the comment on findings input to the sentence display region 52, that is, the content of the interpretation report. In this case, by analyzing the character string included in the interpretation report by using the technique of natural language processing, the region-of-attention specifying unit 23 extracts, as character information, information representing features of the lesion such as the position, type, and size of the lesion included in the interpretation report.

The natural language processing is a series of techniques for causing a computer to process a natural language that humans use on a daily basis. By the natural language processing, it is possible to divide a sentence into words, analyze the syntax, analyze the meaning, and the like. The region-of-attention specifying unit 23 acquires character information and specifies the region of attention by dividing the character string included in the interpretation report into words and analyzing the syntax by using the technique of natural language processing. For example, in a case where the sentence of the interpretation report is “A nodule of 1 cm in size is found in the upper lobe of the right lung”, the region-of-attention specifying unit 23 acquires the terms “right lung”, “upper lobe”, “nodule”, and “1 cm” as character information. Then, the region-of-attention specifying unit 23 specifies the region of attention based on the acquired character information. For example, in a case where the character information is “right lung”, “upper lobe”, “nodule”, and “1 cm”, the nodule in the upper lobe of the right lung is specified as a region of attention.

In this case, in the tomographic image 30A shown in FIG. 4, the nodule included in the right lung 32 is specified as a region of attention. In a case where the abnormal shadow specified as a region of attention is only the nodule in the right lung included in the tomographic image 30A, the non-attention region-of-interest specifying unit 24 specifies regions of interest other than the nodules in the right lung included in the tomographic images 30A to 30H as non-attention regions of interest.

The information acquisition unit 21 may acquire an interpretation report saved in the report DB 8 for the target medical image GO, and analyze the acquired interpretation report to specify an interpreted abnormal shadow and specify a region of attention.

For example, it is assumed that the descriptions in the acquired interpretation report are “I compared with the chest CT performed last time on Jan. 1, 2010. A solid nodule of μ35×28 mm in size is found in the right lung S1. The boundary is unclear with frosted glass shadows on the marginal portion. A pleural invagination is also seen. Calcification and cavities are not included. I think it is a primary lung cancer. A lymph node swollen to μ1.4 cm is found around the B1 bronchi in the right hilum. No pleural effusion is found. Right kidney stone is found. No swollen lymph nodes are found in the abdomen. No ascites is found”. By analyzing such an interpretation report, analysis results of “μ35×28 mm nodule in the right lung S1”, “μ1.4 cm lymphadenopathy around the B1 bronchi”, “no pleural effusion”, “right kidney stone”, “no abdominal lymphadenopathy”, and “no ascites” are obtained.

In this case, the region-of-attention specifying unit 23 may specify the region of interest related to the analysis result among the regions of interest detected by the analysis unit 22 as the region of attention. In addition, the non-attention region-of-interest specifying unit 24 may specify the region of interest that is not related to the analysis result among the regions of interest detected by the analysis unit 22 as the non-attention region of interest.

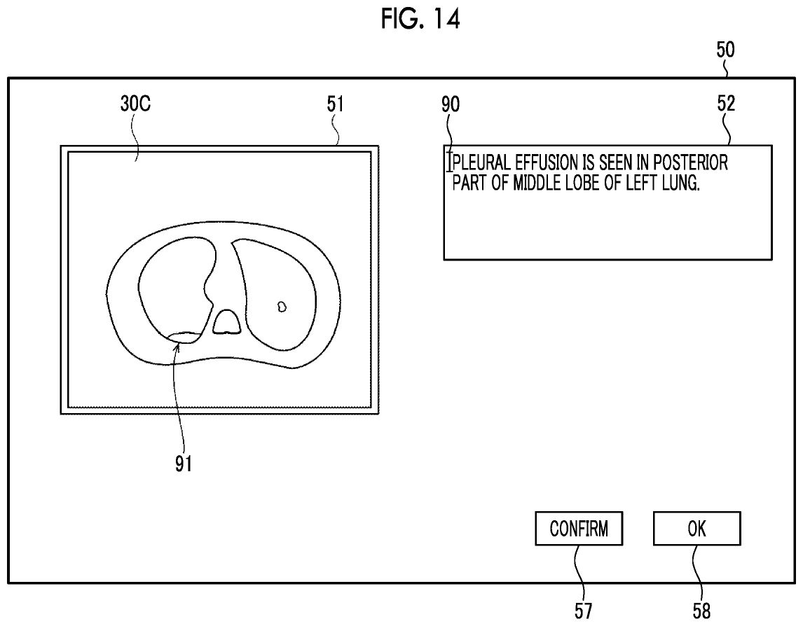

In addition, the region-of-attention specifying unit 23 may specify the region of attention based on the position of the cursor during the input of the comment on findings to the sentence display region 52 during interpretation of the target medical image GO by the radiologist. For example, as shown in FIG. 14, for the tomographic image 30C displayed in the image display region 51, a mark or the like is not added to the tomographic image 30C, but it is assumed that the sentences being input in the sentence display region 52 are “A pleural effusion is seen in the posterior part of the middle lobe of the left lung. A nodule of 1 cm in size is seen in the right lung”. Further, in the sentence display region 52, it is assumed that a cursor 90 is positioned before the character “pleural effusion”. In this case, the region-of-attention specifying unit 23 specifies the region of a pleural effusion 91 included in the tomographic image 30C as the region of attention. In this case, the analysis result for the target medical image GO may be stored in the storage 13 after the analysis process is executed in advance by the analysis unit 22. The region-of-attention specifying unit 23 specifies the region of attention in the tomographic image 30C being displayed based on the character at the position of the cursor 90 in the sentence display region 52 and the analysis result by the analysis unit 22. The position of the cursor 90 is an example of an operation of the user.

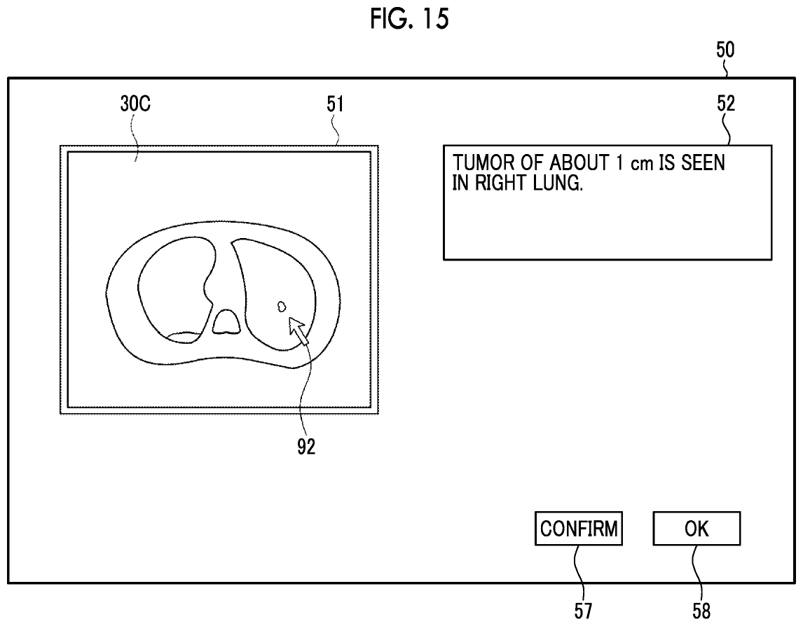

In addition, the region-of-attention specifying unit 23 may specify the region of attention based on the position of the pointer on the target medical image GO displayed in the image display region 51 during interpretation of the target medical image GO by the radiologist. For example, as shown in FIG. 15, it is assumed that a pointer 92 is positioned at the position of the nodule in the right lung included in the tomographic image 30C displayed in the image display region 51. In this case, the region-of-attention specifying unit 23 specifies the nodule region in the right lung included in the tomographic image 30C as the region of attention. In this case, the analysis result for the target medical image GO may be stored in the storage 13 after the analysis process is executed in advance by the analysis unit 22. The region-of-attention specifying unit 23 specifies the region of attention in the tomographic image 30C being displayed based on the position of the pointer 92 in the tomographic image 30C being displayed and the analysis result by the analysis unit 22. The position of the pointer 92 is an example of an operation of the user.

In addition, the region-of-attention specifying unit 23 may specify the region of attention based on the paging operation of the target medical image GO displayed in the image display region 51 during interpretation of the target medical image GO by the radiologist. The paging operation is an operation of sequentially switching tomographic images displayed in the image display region 51. Here, in a case where the tomographic image to be displayed is switched, the radiologist switches the tomographic image by a relatively early paging operation in a case where a disease is not present. On the other hand, in a case where a disease is found in a tomographic image, a paging operation becomes relatively slow in the vicinity of the tomographic image in which the disease is found, the tomographic images are switched back and forth, or a specific tomographic image is displayed for a relatively long period of time.

Therefore, the region-of-attention specifying unit 23 detects that, in the paging operation using the input device 15 by the radiologist, the paging operation becomes relatively slow, the tomographic images are switched back and forth, or the display is performed for a relatively long period of time, and specifies an abnormal shadow included in the tomographic image displayed at the time of detection as a region of attention. For example, in the tomographic images 30A to 30H shown in FIG. 4, in a case where the tomographic image 30C is displayed for a longer period of time as compared with other tomographic images, the region-of-attention specifying unit 23 specifies the pleural effusion in the left lung and the nodule in the right lung included in the tomographic image 30C as regions of attention. In this case, the analysis result for the target medical image GO may be stored in the storage 13 after the analysis process is executed in advance by the analysis unit 22. The region-of-attention specifying unit 23 specifies the region of attention in the tomographic image 30C being displayed based on the analysis result by the analysis unit 22 in the tomographic image 30C that has been displayed for a longer period of time as compared with other tomographic images. The paging operation is an example of an operation of the user.

In addition, the region-of-attention specifying unit 23 may specify the region of attention based on the line of sight of the radiologist during interpretation of the target medical image GO by the radiologist. In this case, a sensor for detecting the line of sight is provided on the display 14, and the line of sight of the radiologist with respect to the tomographic image being displayed on the display 14 is detected based on the detection result of the sensor. For example, in a case where the position of the line of sight is at the nodule in the right lung during the display of the tomographic image 30C, the region-of-attention specifying unit 23 specifies the nodule region in the right lung included in the tomographic image 30C as the region of attention. In this case, the analysis result for the target medical image GO may be stored in the storage 13 after the analysis process is executed by the analysis unit 22 in advance. The region-of-attention specifying unit 23 specifies the region of attention in the tomographic image 30C being displayed based on the detected line of sight of the radiologist and the analysis result by the analysis unit 22. The line of sight is an example of an operation of the user.

On the other hand, in a case where the target medical image GO is a CT image, gradation conditions are set so as to have an appropriate density and contrast such that the target organ can be easily interpreted, and the image is displayed on the display 14. The gradation condition is a window value and a window width in displaying the target medical image GO on the display 14. The window value is a CT value that is the center of a part to be observed in the gradation that can be displayed by the display 14. The window width is a width between a lower limit value and an upper limit value of the CT value of the part to be observed. For example, in a case where the lung field condition is set as the gradation condition such that the lung is easily observed, the window value is the CT value of the lung, and the window width is the lower limit value and the upper limit value of the CT value that make it easier to see the lung. In a case where the lung field condition is set as the gradation condition, the target medical image GO in which the abnormal shadow of the lung can be easily interpreted can be displayed on the display 14. The window value and the window level are examples of a display method.

Here, in a case where a plurality of organs are included in the target medical image GO, there are many cases where the other organs are not interpreted in a case where a gradation condition for easily interpreting a specific organ is set. Therefore, the region-of-attention specifying unit 23 may acquire the gradation condition of the target medical image GO and specify the region of attention in accordance with the gradation condition. For example, in a case where the lung field condition is set as the gradation condition for the target medical image GO, all the abnormal shadows included in the lung in the target medical image GO may be specified as regions of attention. In this case, the non-attention region-of-interest specifying unit 24 may specify the region of interest specified by the analysis unit 22 in the liver as the non-attention region of interest. The gradation condition is an example of a display method.

In addition, in a case where the target medical image GO is a CT image, the CT image is reconstructed by an appropriate reconstruction method in which the target organ can be easily interpreted. Reconstruction is a process performed in a case where a CT image is generated from a projection image acquired by imaging a subject with a CT apparatus. Examples of the reconstruction method include a reconstruction method in which the lungs can be easily observed, a reconstruction method in which the liver can be easily observed, and the like.

Here, in a case where a plurality of organs are included in the target medical image GO, there are many cases where the other organs are not interpreted in a case where the target medical image GO is generated by a reconstruction method in which a specific organ can be easily interpreted. Therefore, the region-of-attention specifying unit 23 may acquire the reconstruction method of the target medical image GO and specify the region of attention in accordance with the reconstruction method. For example, in a case where the reconstruction method in which the lungs can be easily observed is used in generating the target medical image GO, all the abnormal shadows included in the lung in the target medical image GO may be specified as regions of attention. In this case, the non-attention region-of-interest specifying unit 24 may specify the region of interest specified by the analysis unit 22 in the liver as the non-attention region of interest. The reconstruction method is an example of a display method.

In addition, the region-of-attention specifying unit 23 may detect an organ included in the tomographic image being displayed in a case where the target medical image GO is displayed on the display 14, and specify an abnormal shadow included in the detected organ as the region of attention. In this case, the non-attention region-of-interest specifying unit 24 may specify the region of interest specified in the organ that the region-of-attention specifying unit 23 did not detect in the target medical image GO as a non-attention region of interest. For example, in a case where the region-of-attention specifying unit 23 detects a lung from a tomographic image being displayed, the region-of-attention specifying unit 23 specifies an abnormal shadow included in the lung as a region of attention. In addition, the non-attention region-of-interest specifying unit 24 may specify the region of interest specified in an organ other than the lung included in the target medical image GO as a non-attention region of interest.

In addition, in each of the above embodiments, the non-attention region-of-interest specifying unit 24 specifies, as non-attention regions of interest, regions of interest other than the regions of attention specified by the region-of-attention specifying unit 23 among the regions of interest detected by the analysis unit 22, but the present disclosure is not limited thereto. The analysis unit 22 detects an abnormal shadow based on the degree of certainty that the specified region is an abnormal shadow using by the learning model 22A. Therefore, among the regions of interest other than the regions of attention specified by the region-of-attention specifying unit 23, the regions of interest in which the degree of certainty is equal to or greater than a predetermined threshold value Th1 may be specified as non-attention regions of interest. Thus, it becomes possible to perform the secondary interpretation in addition to the primary interpretation for the region of interest in which the possibility of a disease is high. The degree of certainty is an example of a feature amount.

In addition, the learning model 22A in the analysis unit 22 may be configured to derive a degree of malignancy of the abnormal shadow, and the non-attention region of interest may be specified according to the degree of malignancy. That is, among the regions of interest other than the regions of attention specified by the region-of-attention specifying unit 23, the non-attention region-of-interest specifying unit 24 may specify, as non-attention regions of interest, the regions of interest in which the degree of malignancy output from the learning model 22A is equal to or greater than a predetermined threshold value Th2. Thus, it becomes possible to perform the secondary interpretation in addition to the primary interpretation for the region of interest in which the possibility of a disease is high. The degree of malignancy is an example of a feature amount.

Further, in the above embodiments, the analysis unit 22 detects the region of interest from the target medical image GO and derives the annotation, but the present disclosure is not limited thereto. The target medical image GO may be analyzed by an analysis device provided separately from the medical image processing apparatus 20 according to the present embodiment, and the analysis result acquired by the analysis device may be acquired by the information acquisition unit 21. Also, there are cases where the medical care WS 4 can analyze medical images. In such a case, the information acquisition unit 21 of the medical image processing apparatus 20 according to the present embodiment may acquire the analysis result acquired by the medical care WS 4. Further, in a case where the analysis result is registered in the image database 6 or the report database 8, the information acquisition unit 21 may acquire the analysis result from the image database 6 or the report database 8.

In addition, in the first embodiment, the non-attention region-of-interest specifying unit 24 specifies a non-attention region of interest that is a region of interest for an organ different from the organ related to the region of attention among the regions of interest detected by the analysis unit 22 from the target medical image GO. In the second embodiment, the non-attention region-of-interest specifying unit 24 specifies, as a non-attention region of interest, a region of interest for a disease different from the disease related to the region of attention among the regions of interest detected by the analysis unit 22 from the target medical image GO. However, the non-attention region-of-interest specifying unit 24 may specify, as non-attention regions of interest, both a region of interest for an organ different from the organ related to the region of attention and a region of interest for a disease different from the disease related to the region of attention among the regions of interest detected by the analysis unit 22 from the target medical image GO.

Further, in each of the above embodiments, the technique of the present disclosure is applied in the case of creating an interpretation report using a medical image with lung or liver as the diagnosis target, but the diagnosis target is not limited to lung or liver. In addition to the lung, any part of a human body such as a heart, brain, kidneys, and limbs can be used as a diagnosis target. In this case, the diagnostic guideline according to the diagnosis target part may be acquired, and the corresponding portion corresponding to the item of the diagnostic guideline in the interpretation report may be specified.

Further, in each of the above embodiments, for example, as hardware structures of processing units that execute various kinds of processing, such as the information acquisition unit 21, the analysis unit 22, the region-of-attention specifying unit 23, the non-attention region-of-interest specifying unit 24, the display control unit 25, the interpretation report creation unit 26, and the communication unit 27, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example where a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

What is claimed is:

1. A medical image processing apparatus comprising at least one processor,
wherein the processor is configured to:
acquire a result of detecting at least one region of interest included in a medical image, which is detected by analyzing the medical image;
specify a region of attention that a user has paid attention to in the medical image;
specify a non-attention region of interest, which is a region of interest having a structure different from a structure related to the region of attention, among the regions of interest; and
display a result of specifying the non-attention region of interest on a display.

2. The medical image processing apparatus according to claim 1,
wherein the processor is configured to detect at least one region of interest included in a medical image by analyzing the medical image.

3. The medical image processing apparatus according to claim 1,
wherein the processor is configured to display the result of specifying the non-attention region of interest by erasing a result of detecting a region of interest having the structure related to the region of attention among the regions of interest.

4. The medical image processing apparatus according to claim 1,
wherein the processor is configured to specify the region of attention based on an operation of the user during interpretation of the medical image.

5. The medical image processing apparatus according to claim 1,
wherein the processor is configured to specify the region of attention based on a document regarding the medical image.

6. The medical image processing apparatus according to claim 1,
wherein the processor is configured to specify the region of attention based on a method of displaying the medical image during interpretation of the medical image.

7. The medical image processing apparatus according to claim 1,
wherein the processor is configured to display a result of detecting a region of interest having the structure related to the region of attention, the region of interest of which a feature amount derived at a time of detection is equal to or greater than a predetermined threshold value.

8. The medical image processing apparatus according to claim 1,
wherein the region of interest is a region of interest for a plurality of types of diseases.

9. The medical image processing apparatus according to claim 1,
wherein the region of interest is a region of interest for a plurality of types of organs.

10. A medical image processing method comprising:
acquiring a result of detecting at least one region of interest included in a medical image, which is detected by analyzing the medical image;
specifying a region of attention that a user has paid attention to in the medical image;
specifying a non-attention region of interest, which is a region of interest having a structure different from a structure related to the region of attention, among the regions of interest; and
displaying a result of specifying the non-attention region of interest on a display.

11. A non-transitory computer-readable storage medium that stores a medical image processing program causing a computer to execute:
a procedure of acquiring a result of detecting at least one region of interest included in a medical image, which is detected by analyzing the medical image;
a procedure of specifying a region of attention that a user has paid attention to in the medical image;
a procedure of specifying a non-attention region of interest, which is a region of interest having a structure different from a structure related to the region of attention, among the regions of interest; and
a procedure of displaying a result of specifying the non-attention region of interest on a display.
